(19) United States
(12) Patent Application Publication
(10) Pub. No.: US 2022/0105324 A1
(43) Pub. Date: Apr. 7, 2022

(54) VASCULAR ACCESS DILATOR APPARATUS AND METHOD OF USE

(71) Applicant: D & J SOLUTIONS, LLC, Philadelphia, PA (US)

(72) Inventors: GERALD WALTER BRONIEC, Philadelphia, PA (US); DAVID HAMMILL, Philadelphia, PA (US)

(73) Assignee: D & J SOLUTIONS, LLC, Philadelphia, PA (US)

(21) Appl. No.: 17/509,913

(22) Filed: Oct. 25, 2021

Related U.S. Application Data
(63) Continuation-in-part of application No. 16/782,942, filed on Feb. 5, 2020.
(60) Provisional application No. 62/801,377, filed on Feb. 5, 2019.

(51) Int. Cl. A61M 29/02; A61B 17/02; A61M 25/06; A61M 25/09

(52) U.S. Cl. CPC A61M 29/02; A61B 17/0218; A61M 25/09041; A61M 2205/582; A61M 2205/583; A61M 25/0631

(57) ABSTRACT

An improved vascular access dilator apparatus consisting of a wire, a blade with a locking safety mechanism, and a malleable, tapered dilator having a lumen just large enough to accommodate said wire sliding easily in and out. The tapering of said dilator being accomplished by and improved by the joining of straight dilator segments (that decrease in diameter in the direction of the tip of the dilator) by angled segments located between each straight segment. In one embodiment, A safety sheath covers the blade once an incision in the dermis is made, thereby protecting both the patient and caregiver.

### CROSS REFERENCE TO RELATED APPLICATIONS

This is a continuation in part application based on pending U.S. Utility application Ser. No. 16/782,942 that claimed priority from U.S. Provisional Application No. 62/801,377 filed on Feb. 5, 2019, all of which are incorporated by reference herein.

### FIELD OF THE INVENTION

The present invention relates to an improved vascular access dilator apparatus for medical use and the method of use thereof.

### BACKGROUND

Vascular access of a peripheral venous or arterial insertion site via Seldinger technique can be challenging and dangerous for both the patient and caregiver. Obtaining vascular access, however, is a requirement for the insertion of a flexible, sterile thin plastic tubes known as catheters as treatment devices. Most of all vascular access enables an effective method of drawing blood or delivering medications, blood products, or nutrition into a patient's bloodstream.

This access can also be useful for the placement of cannulas such as arterial and/or venous which can remove deoxygenated venous blood and deliver oxygenated arterial blood. In the world of medical devices there are quite a few options for access but unfortunately none offer a complete apparatus that encompasses all the parts necessary for safe and effective access.

Although it is a fairly straightforward procedure, factors such as body habitus, peripheral artery disease, and ongoing cardiac arrest all complicate vascular access achieval. After vascular access is achieved, a small incision in the dermis of the patient's skin is performed so that the site can accommodate the cannula and the series of dilators that facilitate the cannula's insertion via stepwise dilation.

During cannula insertion Seldinger technique is utilized for vascular access. This is followed by small incision in the dermis and then a series of dilations in a stepwise manner allows the artery or vein to accommodate the cannula that will be placed. This device allows for a quick and simple process by the use of a one-time dilator that has a small blade integrated into the system. Instead of serial dilation and reaching for multiple instruments which requires more time and tools.

An example of a dilator for the access of a peripheral venous or arterial insertion can be seen in U.S. Patent Application number US 2015/0297875A1, which describes the device and method capable of gaining peripheral access. This device requires multiple steps and pieces which in the setting of needing access means a crowded and messy field, along with multiple steps and processes. In an emergency setting the whole scenario becomes exponentially more complicated, dangerous, unsafe for patients along with caregivers, and much more cumbersome with the additional responsibility of grim outcome for the patient.

Therefore the need for an improved vascular dilator apparatus exists. There is also a need for a complete apparatus that already includes within its content the pieces needed for safe and effective access.

### SUMMARY OF THE INVENTION

One embodiment of the present invention comprises a novel vascular dilator apparatus consisting of the following: a 0.35″ wire, a blade with a locking safety mechanism, a dilator (that in one embodiment may be 7 cm long) that is malleable. The dilator has a lumen just large enough for the wire to slide easily in and out. The safety sheath is intended to cover the blade once an incision in the dermis is made. This protects both the patient and caregiver. This apparatus is intended to enter the body through the fascia and vessel wall therefore dilating the region up to a prescribed size.

A second embodiment discloses an improvement to the dilator construction that is based on the use of angled segments that connect straight segments of the dilator. The improved embodiment provides for a measured, and therefore more accurate, dilation that provides both tactile and visual feedback to the medical professional conducting the vascular access. In this way, an improved dilator of the second embodiment facilitates more accurate insertion and ease of insertion, which together work to provide a method of vascular access that better prevents against vascular injury in the process of vascular access. Additionally, an improved embodiment of the system uses an improved blade device as further described herein.

### BRIEF DESCRIPTION OF THE DRAWINGS

Some embodiments of the present invention are illustrated as an example and not limited by the figures of the accompanying drawings, in which like references may indicate similar elements and in which:

FIG. 1 depicts a side perspective view of a first embodiment of a vascular access dilator apparatus.

FIG. 2 depicts a close up perspective view of the blade, safety device, inserted wire and french sizes used for sizing for said first embodiment.

FIG. 3 depicts a top view of the vascular access dilator apparatus of said first embodiment.

FIG. 4 depicts an underside view of the vascular access dilator apparatus of said first embodiment.

FIG. 5 depicts a close up view of the blade safety device along with the french sizes and the wire that is inserted through the device of said first embodiment.

FIG. 6 is a side view of a dilator of a second embodiment of the instant invention.

FIG. 7 is a cutaway view of the side view of said dilator of FIG. 6.

FIG. 8 is a cutaway view of the side view of said dilator of FIG. 6 that identifies the measured angle from the plane of the angled segments of said dilator.

FIG. 9 is a side view of said dilator of FIG. 6 that indicates the width of the segments of said dilator.

FIG. 10 is a side view of said dilator of FIG. 6 that indicates the length of the segments of said dilator.

FIG. 11 is a perspective view of an improved slidable blade structure for use with the improved dilator of the instant invention.

FIG. 12 is a perspective view of an improved slidable blade structure for use with the improved dilator of the instant invention.

FIG. 13 is a cutaway view of an improved slidable blade structure of the instant invention.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Vascular access using this dilator apparatus are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as a set of exemplifications of the invention, and is not intended to limit the invention to the specific embodiments illustrated by the figures or description below.

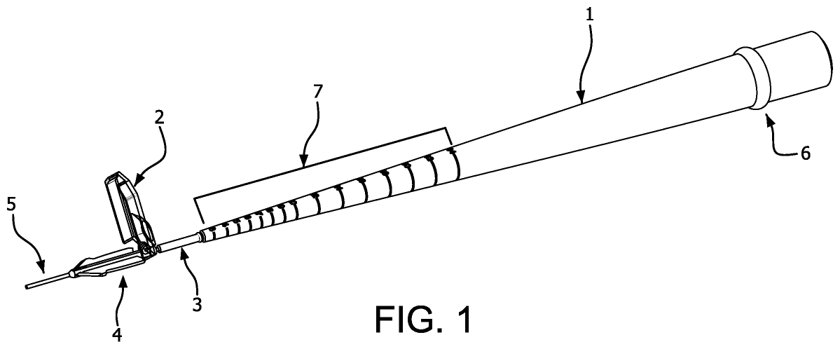

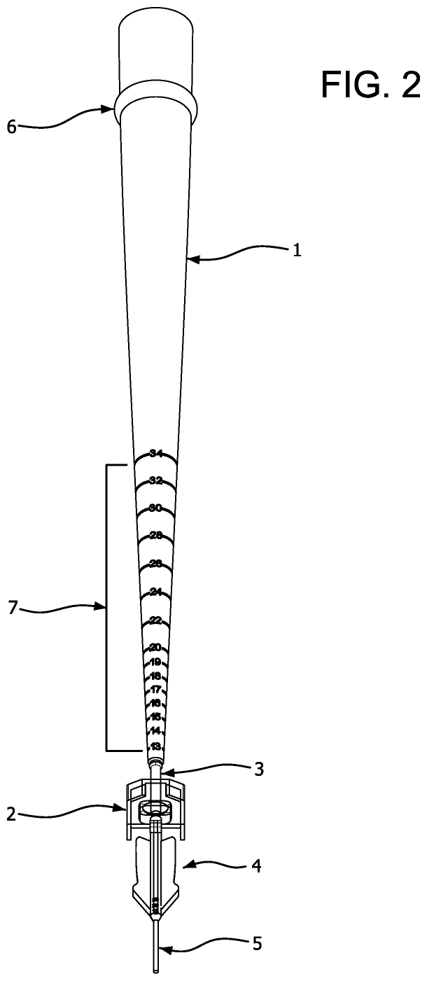

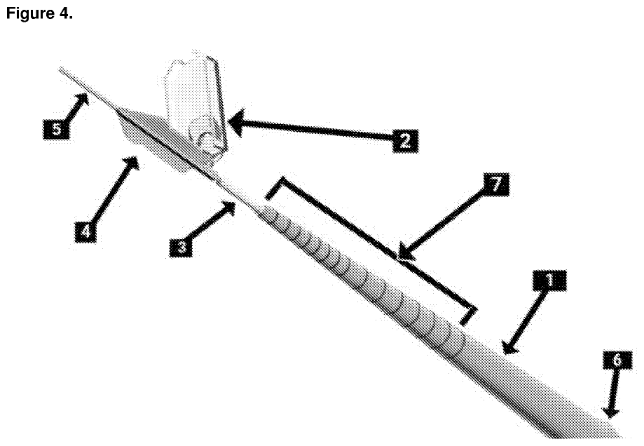

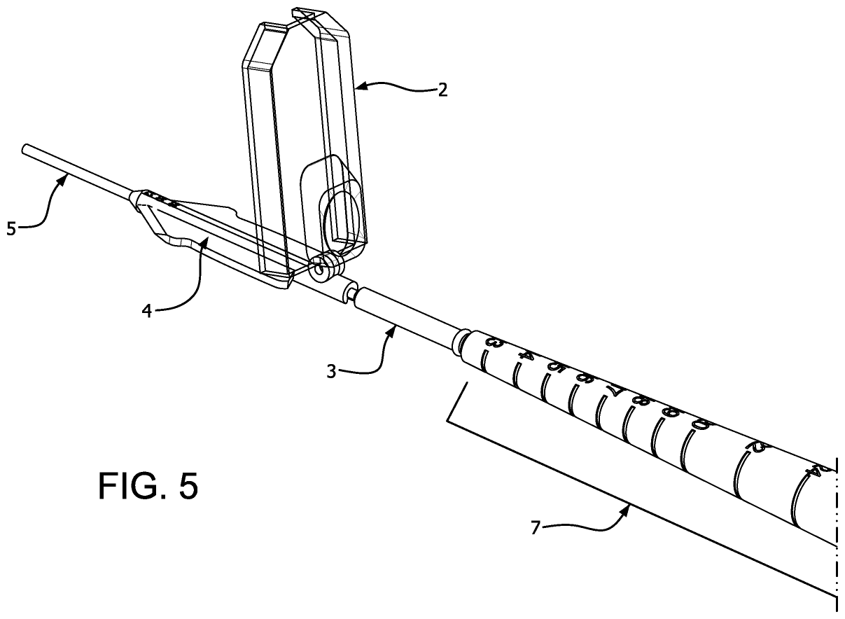

A 21 gauge needle is to be inserted into a patient's vessel. This hollow needle creates a passageway into the targeted vessel, referred to as “access.” FIG. 1 depicts wire 5, which is then inserted through the needle into that patient's bloodstream, either an artery or vein in a first embodiment of the current invention. Once the wire is satisfactorily placed into the lumen of the vessel, the needle is slid over the wire, away from the patient, and off the wire. FIG. 2 shows blade 4, which slides down the wire to make a small incision in the patient's dermis to facilitate dilation. Additionally, FIGS. 2 and 5 depict markings that correlate with french sizes 8 for vessel cannula insertion. After the incision is made, the blade is withdrawn from the patient and covered by safety cover 2 as seen in FIG. 2. Lastly, the covered blade is removed from the wire and disposed per facility procedure.

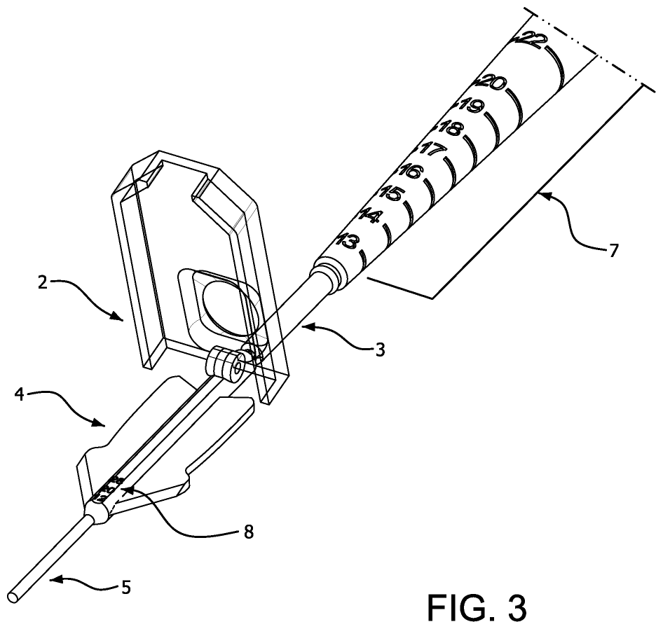

Dilator 1 then slides down wire and is inserted into the lumen of the patient's vessel to the desired french size as shown in FIGS. 3 and 4. Sizes shown in FIG. 3 refer to the depth that the dilator can be inserted into patient to achieve desired dilation size. French sizes 7 are printed on the side of the cannula with circumferential markings so the user can properly dilate. Additionally, band 6 can be placed onto the prescribed size on dilator 1 to prevent over dilation.

Once dilation with apparatus is complete the dilator is removed from the wire. Desired cannula is placed along wire, inserted into patient's vessel, and said wire is removed.

An improved dilator of a second embodiment is disclosed in FIG. 6 through FIG. 10. FIG. 6 shows a side view of a second embodiment of such an improved dilator 10. In FIG. 6 the insertable section of a non-rigid dilator 10 (having a length designated 30, which, in the preferred embodiment, is 40 cm to 45 cm) has angled segments 11, 12, 13, and 14 that join together straight segments 21, 22, 23, and 24, with segment 24, having the smallest diameter of the straight segments, culminating in tip 15 of the dilator assembly.

FIG. 6 indicates that the entire length of the dilator assembly 50 that includes a section of variable length that is not intended to be inserted into a patient's vessel. The rigidity of the tip and the tail of the dilator, as measured by a durometer, may vary such that the tail of the dilator may be more rigid that the tip.

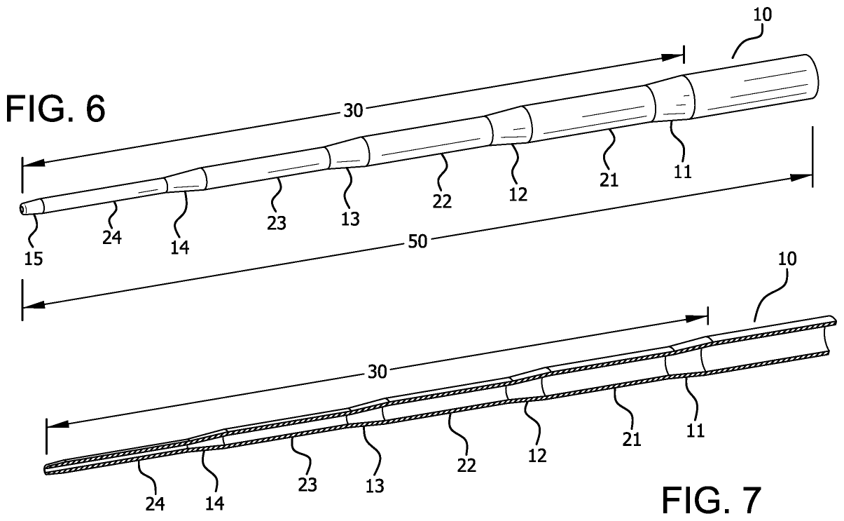

FIG. 7 is a cutaway view of the side view of FIG. 6 also labelling as 30 the length of the insertable section of dilator 10.

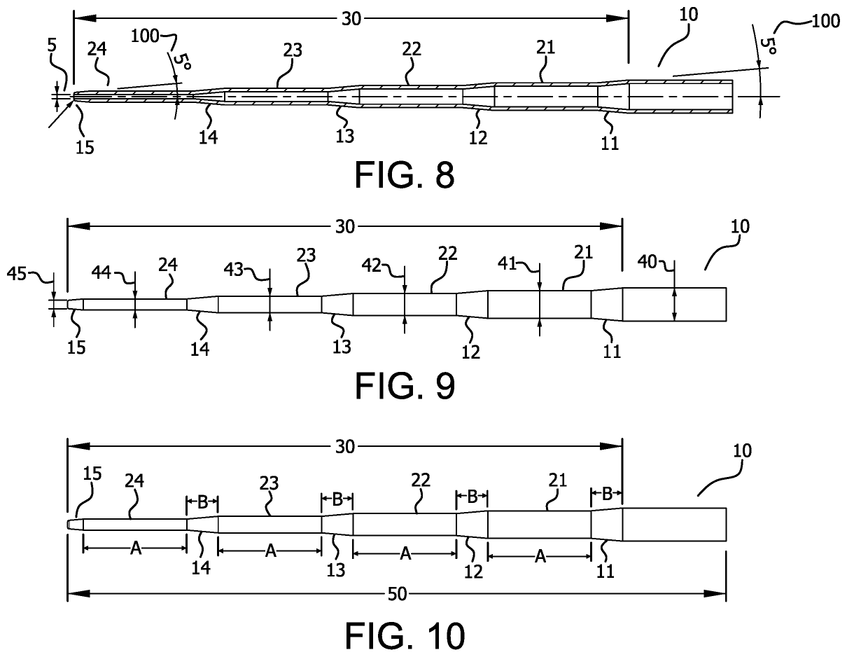

FIGS. 8, 9, and 10 are illustrative of angular displacements and linear dimensions of each of the segments of the insertable portion of length 30 of the dilator assembly 10. In FIG. 8 equal angles 100 show that the each one of the angled segments 11, 12, 13, and 14 of dilator assembly 10 are set on an acute 5 degree angle from the preceding straight section, that is, respectively, 21, 22, 23, and 24. FIG. 8 also shows wire 5, in a preferred embodiment being of 1 cm in diameter, exiting from tip 15 of the insertable section 10.

FIG. 9 identifies the diameter of each of the straight segments of insertable section of length 30 of the dilator assembly in a preferred embodiment. Straight segment 21 having a diameter 41 of 6.67 cm; straight segment 22 having a diameter 42 of 5.33 cm; straight segment 23 having a diameter 43 of 4.00 cm; and straight segment 24 having a diameter 44 of 2.67 cm. The tail, that is, the section of dilator assembly that is not intended to be inserted into a patient's vessel has a diameter 40 of 8 cm, the tip 15 of the dilator has a diameter 45 of 2.06 cm.

FIG. 10 identifies the length of each of the straight segments and each of the angled segments of insertable section of length 30 of the dilator assembly in a preferred embodiment. Straight segments 21, 22, 23, and 24, also referred to as landing zones, are each have a length A of 6.0 cm. Angled segments 11, 12, 13, and 14, also referred to as transition zones, are each have a length B of 2.5 cm. The section of dilator assembly that is not intended to be inserted into a patient's vessel, also referred to as the remainder zone, has a length in a preferred embodiment of 10.0 cm. Tip 15, or the initial zone, has a length of 1.0 cm. In a preferred embodiment, the insertable section has a length 30 of 41.0 cm and the entire length 50 of the dilator assembly is 51 cm.

The second embodiment of a dilator assembly as disclosed herein describes an improvement to dilator construction that is based on the use of angled segments that connect straight segments of the dilator. In preferred embodiment, each successive angled segment is set on an acute 5 degree angle from the plane of each straight segment creating a series of landing zones between each transition zone, that is, within the length of each angled segment. In the method of using such a second embodiment of a dilator assembly, the medical professional using said assembly proceeds by inserting a hollow needle into the vessel of a patient. Then a wire is inserted longitudinally into said needle and said needle is retracted from the vessel site. The medical professional proceeds by sliding a blade on said wire in the direction of said vessel, which blade is used to make an incision in the dermis of the patient surrounding his or her vessel. The blade is withdrawn and covered with a safety cover, before disposing of said covered blade, thereby preventing any accidental cutting of the patient or the medical professional performing the method. The professional proceeds to position the dilator of this second embodiment on said wire in the direction of said vessel. With the straight segments of the insertable portion of this dilator assembly being designed to have decreasing diameters in the direction of said vessel and having each straight segment to the other straight segments angled segments, the method of then inserting the dilator assembly into said vessel to the depth of the desired straight segment is facilitated by feedback experienced by a medical professional during insertion that is based on tactile feel as the landing zones, that is, the straight segments, are properly and safely inserted into the vessel for introduction of a treatment device. Additionally, the medical professional is aided by visual feedback as the landing zones are seen by the medical professional as the insertion progresses. This improved method using this improved embodiment of the dilator assembly provides for a measured, and therefore more accurate, dilation through both tactile and visual feedback accessible to the medical professional conducting the vascular access process. In this way, an improved dilator of the second embodiment facilitates more accurate insertion and ease of insertion, which together work to provide a method of vascular access that better prevents against vascular injury in the process of vascular access.

Another embodiment of the system and method disclosed herein is described as an apparatus comprising a non-rigid vascular dilator with an interior and an exterior surface wherein the interior surface consists of a variable diameter lumen contiguous along its long axis. The exterior surface being variable in diameter whereby the diameter of one end (the tip) is less than one half as large than that of its other end (the tail). The exterior surface is configured such that its diameter, at more than one point along its length (in between the tip and the tail), is greater than twice the diameter of the tip yet less than that of the tail. The exterior surface is configured with multiple segments each consisting of a separate uniform diameter that may vary in length. The rigidity of the exterior surfaces may be variable at multiple segments along its axis and are configured with a continuously unobstructed lumen. At least one end of its lumen is angled. The system has a pointed steel blade consisting of a top and a bottom surface with more than one cutting edge on its sides. That blade is slidably affixed to one or more channels inside of a safety handle. The blade device has a uniform semicircular channel recessed into its top surface along its entire length configured to receive a surgical wire larger than 0.020 inches and has mechanism for manually advancing and retracting said blade from said handle along said wire. In one embodiment, the apparatus (or a part of it) is magnetized. In another embodiment, the dilator handle has a textured surface capable of being sterilized. Using this system embodiment defines a method for the controlled precise puncture of a blood vessel that contains a surgical wire by removably attaching a surgical blade to a surgical wire whereby said blade is then slidable along said wire. An additional embodiment is disclosed as a dilator assembly with a tapered tip hollow steel needle, a surgical wire, a tissue incision device with a recessed channel on one side of a steel blade, and a vascular dilator with more than one external diameter size separated by 5 degree angular transitions. The dilator assembly is serializable. Another embodiment of the method disclosed herein has a step of inserting a vascular dilator placed over a wire into human tissue utilizing axial and rotational forces applied to said tissue through the manipulation of a single shaped dilator with a variable diameter.

This disclosure of one or more of the secondary embodiments and methods is to be considered as a set of exemplifications of the invention, and is not intended to limit the invention to the specific embodiments illustrated by the figures or descriptions herein.

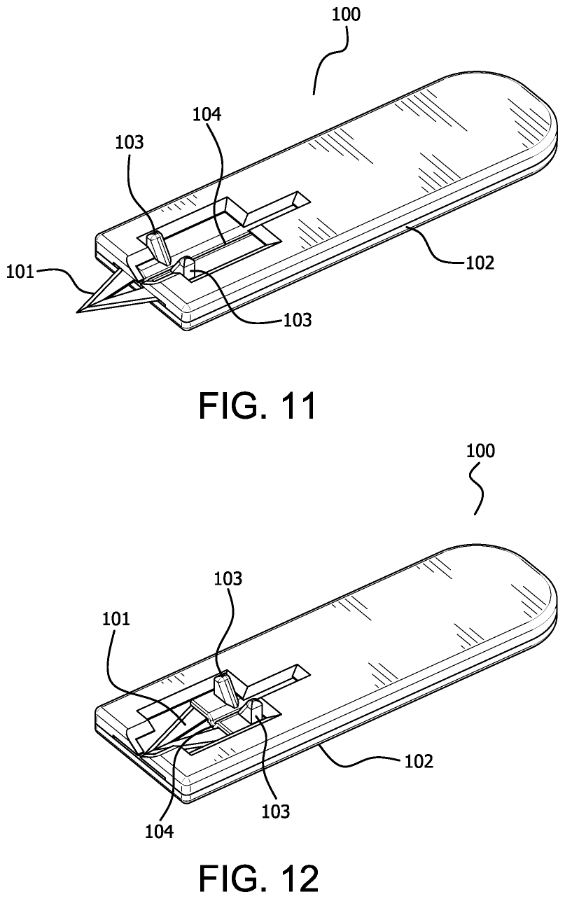

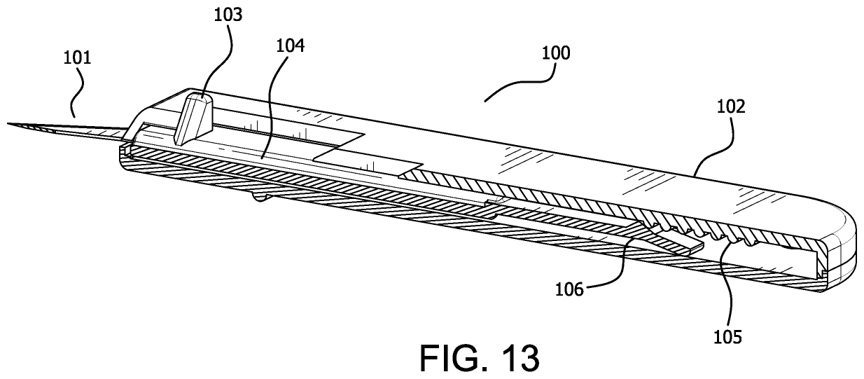

An improved structure for a slidable blade device 100 for use with a dilator of the present invention is shown in perspective view in FIG. 11 and FIG. 12. FIG. 11 shows the sharped end of elongated blade 101 protruding from housing structure 102 of said device 100, while in FIG. 12 said sharpened end of said elongated blade 101 has been retracted by a user by means of retractor arms 103. Said retraction allows said sharpened end of said elongated blade 101 to be retracted after use and secured within holder 102 of device 100 for safety purposes. In FIGS. 11 and 12 a recessed channel 104 is shown in structure 102 which channel 104 provides a guide for sliding blade structure 100 down wire 5 of the preferred embodiment in the nature of a monorail type arrangement. FIG. 13, a cutaway view of structure 100, shows the manner in which said elongated blade 101 is situated in housing structure 102 in a preferred embodiment. In FIG. 13 sawtoothed assembly 105 is shown that engages a cam 106 located on the top of elongated blade 101 as the said elongated blade is retracted allowing the retraction (or extension) of said elongated blade within said housing by use of said retractor arms 103.

This disclosure of an improved embodiment of a blade device is to be considered as a set of exemplifications of the invention, and is not intended to limit the invention to the specific embodiments illustrated by the figures or descriptions herein.

What is claimed is:

1. A vascular access dilator apparatus comprising:
a hollow needle;
a wire insertable longitudinally into said needle;
a blade slidable on said wire;
a malleable, tapered dilator insertable directionally in line with said wire, said dilator having a tip; and
a safety cover retractable from said blade;
whereby said dilator is comprised of a series of straight segments that decrease in diameter in the direction of said tip which straight segments are connected by angled segments located between each of said straight segments.

2. The vascular access dilator apparatus of claim 1 in which said malleable, tapered dilator has French sizes printed thereon.

3. The vascular access dilator apparatus of claim 1 in which each of said angled segments is tapered by an angle of 5° in the direction of said tip.

4. In a vascular access dilator system having a needle, a wire, a blade, and a dilator having a tip, the improvement comprising:
a safety cover retractable from said blade,
whereby safe disposal of such blade after use is obtained and whereby said dilator is malleable and is comprised of a series of straight segments that decrease in diameter in the direction of said tip which straight segments are connected by angled segments located between each of said straight segments.

5. In the improvement of claim 4, each of said angled segments being tapered by an angle of 5° in the direction of said tip.

6. A method for introduction of a treatment device into a body vessel and safe disposal of apparatus therefor by a medical professional comprising the steps of:
inserting a hollow needle into said vessel;
inserting a wire longitudinally into said needle;
retracting said needle;
sliding a blade on said wire in the direction of said vessel;
using said blade to make an incision in the dermis surrounding said vessel;
withdrawing said blade;
covering said blade with a safety cover retractable therefrom;
disposing of said covered blade;
sliding a dilator on said wire in the direction of said vessel, said dilator having straight segments having decreasing diameters in the direction of said vessel, which straight segments being connected by angled segments;
inserting said dilator into said vessel to the depth of the desired straight segment using visual and tactile feedback accessible to said medical professional inserting said dilator based related to the visible level of insertion of said straight segment and the feel of insertion as said straight segment is inserted following insertion of a preceding angled segment,
whereby said dilator is placed in said vessel in a safe manner for proper dilation for introduction of a treatment device.

7. In the method of claim 6, each of said angled segments being tapered by an angle of 5° in the direction of said vessel.

8. A vascular access dilator apparatus comprising:
a hollow needle;
a wire insertable longitudinally into said needle;
a blade device slidable on said wire; and
a malleable, tapered dilator insertable directionally in line with said wire, said dilator having a tip;
whereby said dilator is comprised of a series of straight segments that decrease in diameter in the direction of said tip which straight segments are connected by angled segments located between each of said straight segments.

9. The vascular access dilator apparatus of claim 8 in which said malleable, tapered dilator has French sizes printed thereon.

10. The vascular access dilator apparatus of claim 8 in which each of said angled segments is tapered by an angle of 5° in the direction of said tip.

11. The vascular access dilator apparatus of claim 8 in which said blade device comprises:
an elongated blade having a sharpened end and having a cam mounted thereon;
a housing having a longitudinal recessed channel; and
retractor means,
wherein said blade is longitudinally retractable withing said housing and said blade device is slidable on a guide wire for use with a vascular dilator.

12. The vascular access dilator assembly of claim of claim 11 in which said retractor means comprises:
retractor arms connected to said elongated blade;
a retraction opening within said housing into which said elongated blade is longitudinally housed; and
a sawtoothed structure within said retraction opening;
wherein said sawtoothed structure engages said cam providing for retraction of said blade using said retractor arms.

13. A blade device for use with a vascular dilator comprising:
an elongated blade having a sharpened end and having a cam mounted thereon;
a housing having a longitudinal recessed channel; and
retractor means,
wherein said blade is longitudinally retractable within said housing and said blade device is slidable on a guide wire for use with a vascular dilator.

14. The blade device of claim 13 in which said retractor means comprises:
retractor arms connected to said elongated blade;
a retraction opening within said housing into which said elongated blade is longitudinally housed; and
a sawtoothed structure within said retraction opening;
wherein said sawtoothed structure engages said cam providing for retraction of said blade using said retractor arms.

15. A method for introduction of a treatment device into a body vessel and safe disposal of apparatus therefor by a medical professional comprising the steps of:
inserting a hollow needle into said vessel;
inserting a wire longitudinally into said needle;
retracting said needle;
sliding a blade device having a sharpened end longitudinally on said wire in the direction of said vessel;
using said sharpened end of said blade device to make an incision in the dermis surrounding said vessel;
retracting said sharpened end into said blade device;
withdrawing said blade device;
sliding a dilator on said wire in the direction of said vessel, said dilator having straight segments having decreasing diameters in the direction of said vessel, which straight segments being connected by angled segments;
inserting said dilator into said vessel to the depth of the desired straight segment using visual and tactile feedback accessible to said medical professional inserting said dilator based related to the visible level of insertion of said straight segment and the feel of insertion as said straight segment is inserted following insertion of a preceding angled segment,
whereby said dilator is placed in said vessel in a safe manner for proper dilation for introduction of a treatment device.

16. In the method of claim 16, each of said angled segments being tapered by an angle of 5° in the direction of said vessel.

17. In the method of claim 16, the blade device comprising:
an elongated blade having a sharpened end and having a cam mounted thereon;
a housing having a longitudinal recessed channel; and
retractor means,
wherein said blade is longitudinally retractable within said housing and said blade device is slidable on a guide wire for use with a vascular dilator.

18. In the method of claim 18, said retractor means comprises:
retractor arms connected to said elongated blade;
a retraction opening within said housing into which said elongated blade is longitudinally housed; and
a sawtoothed structure within said retraction opening;
wherein said sawtoothed structure engages said cam providing for retraction of said blade using said retractor arms.
